(19) United States
(12) Patent Application Publication
(10) Pub. No.: US 2018/0214388 A1
(43) Pub. Date: Aug. 2, 2018

(54) COMPOSITION AND METHOD FOR REDUCING SCARRING

(71) Applicant: Jeptha N. COLE, Jackson, MS (US)

(72) Inventor: Jeptha N. COLE, Jackson, MS (US)

(21) Appl. No.: 15/746,768

(22) PCT Filed: Jul. 26, 2016
(86) PCT No.: PCT/US2016/044109

§ 371 Date: Jan. 22, 2018

Related U.S. Application Data
(60) Provisional application No. 62/197,459, filed on Jul. 27, 2015.

(51) Int. Cl. A61K 31/05; A61P 17/02; A61K 31/522; A61K 33/06; A61K 9/00; A61K 45/06

(52) U.S. Cl. CPC A61K 31/05; A61P 17/02; A61K 45/06; A61K 33/06; A61K 9/0014; A61K 31/522

(57) ABSTRACT

A kit for reducing scarring comprises a first composition including resveratrol and a second composition including a primary agent selected from the group consisting of caffeine, caffeine metabolites, and mixtures thereof. A method of reducing scarring comprises applying to a wound or incision a first composition including resveratrol and applying to the wound or incision a second composition including a substance selected from the group consisting of caffeine, caffeine metabolites, and mixtures thereof.

### BACKGROUND

Wound healing is a complex process and involves the regulation of numerous cellular functions, including the interactions of fibroblasts/fibrocytes, osteoblasts, chondrocytes, endothelial cells, inflammatory cells, epithelial cells and smooth muscle cells with the extracellular matrix. Normal healing results in scar formation in humans. However, it is well known that certain animals, and even the human fetus, are capable of regenerative healing of wounds that is indistinguishable from surrounding skin.

Although the intricate details of wound healing are still being discovered, the process follows along a typical timeline having four phases:

Hemostasis phase—This phase includes vasoconstriction lasting for the first 5-10 minutes after an injury.

Inflammation phase—This phase includes vasodilation and the cellular response by inflammatory macrophages, neutrophils and fibroblasts. Neutrophils undergo cannibalization to produce transforming growth factor beta (TGF-β), which stimulates production of Type I collagen (the mature collagen present in normal skin) and stimulates fibroblasts to myofibroblasts mediated by hyaluronic acid and epidermal growth factor receptor (EGFR). Bacteria, foreign particles and damaged cells are removed from the wound. Vasodilation starts about 10 minutes after the initial injury and the cellular response typically starts 30 minutes after the initial injury. Keratinocytes detach from the basement membrane and migrate to cover the exposed wound and connective tissue, and the wound clot is replaced with epithelial cells and granulation tissue (Type III collagen). Differentiating keratinocytes also produce TGF-β1. The cellular response may last 7 to 8 days.

Proliferation phase—This phase includes re-epithelialization of the wound, fibroplasia/collagen synthesis and wound contraction. During this phase skin cells multiply and spread, covering the wound. Re-epithelialization typically starts 24 hours after the injury. Fibroplasia typically starts 3 to 4 days after the injury. TGF-β1 is up-regulated during this phase. TGF-β1 and connective tissue growth factor (CTGF) cause over-activation of fibroblasts and collagen deposition. Myofibroblasts (present in granulation tissue) express alpha-smooth muscle actin and are responsible for wound contraction, which typically starts 7 days after the injury.

Remodeling phase—This phase includes scar/collagen remodeling. The newly formed collagen matrix becomes cross linked and organized starting about 3 weeks from wound initiation and lasting as long as 1 year.

TGF-β is a potent Type II cytokine expressed by epithelial and fibroblast cells. TGF-β is a prototypical fibrogenic cytokine responsible for fibroblast proliferation, activation and fibrosis in wound healing. TGF-β cooperates with CTGF to sustain fibrosis and to exacerbate extracellular matrix production in association with other fibrosis-inducing conditions. TGF-β exists in three forms, TGF-β1, TGF-β2 and TGF-β3, with TGF-β1 having the strongest up-regulation of fibroblasts. The TGF-β/SMAD signaling pathway is one of the key pathways involved in fibrosis.

CTGF is a matricellular protein of the CCN family of extracellular matrix-associated heparin-binding proteins. CTGF is associated with wound healing and virtually all fibrotic pathology. Over-expression of CTGF in fibroblasts promotes fibrosis in the dermis, kidney and lung, while deletion of CTGF in fibroblasts and smooth muscle cells reduces bleomycin-induced skin fibrosis. CTGF is regulated in a TGF-β/Smad3 dependent fashion.

As compared with normal skin, a scar contains an overproduction of Type III and Type I collagens and the mixture is disorganized. The scar itself is not very elastic and is of a different color than normal skin. The scar is also missing the layer of keratinocytes found on normal skin. Furthermore, depending on the depth of the original wound, the scar may be missing the normal underlying layers of muscle, fat, blood vessels and many layers of skin. These missing layers may result in the scar forming a depression compared to the level of the surrounding skin.

Some animals, such as the axolotl, are capable of scar-free healing. In axolotls, there is a substantial reduction in neutrophil infiltration and a relatively long delay in production of new extracellular matrix during scar-free healing. Studies with athymic nude mice indicate that up-regulation in matrix metalloproteinase-9 (MMP-9) throughout the remodeling phase may contribute to scar-free healing. Matrix metalloproteinases (MMPs) are a family of zinc-dependent enzymes capable of degradation of the extracellular matrix and are vital to the remodeling of the matrix and migration of cells. During normal human wound healing MMP-9 degrades the Type IV collagen of the basement membrane, allowing keratinocytes to detach from the basement membrane and migrate to cover the exposed wound and connective tissue.

Humans are capable of scar-free healing in certain situations. The healing of human oral wounds results in little to no scar formation. Oral mucosal wounds show a robust early up-regulation of MMP-1, MMP-2 and MMP-9 at 3 days after the initial injury, while skin wounds show up-regulation at 14 days after the initial injury. The human fetus is also capable of scar-free healing. This may be a result of the fetus lacking a mature immune system or being surrounded by amniotic fluid, which contains high molecular weight hyaluronic acid. High molecular weight hyaluronic acid is known to increase expression of MMP-2 and MMP-9. Although high molecular weight hyaluronic acid application at a wound site can reduce scarring, a scar is nevertheless still formed.

Resveratrol (trans-3,4′,5-trihydroxystilbene), a stilbenoid, is a natural polyphenol present in various plants, some food products, red wine and grapes. Resveratrol possesses anti-inflammatory, anti-carcinogenic and anti-oxidant properties, and has been extensively studied. Huge interest in resveratrol was created when it was discovered that it was able to active the SIRT1 gene, a gene implicated in the life span extension associate with calorie-restricted diets. However, resveratrol is poorly absorbed when consumed as a dietary supplement and is subject to metabolic degradation. Beneficial effects have been difficult to observe in human clinical studies.

Caffeine (1,3,7-trimethylpurine-2,6-dione) is a bitter, white crystalline xanthine alkaloid. Caffeine is found in varying quantities in the seeds, leaves and fruit of some plants, such as the coffee plant, the tea bush and the kola nut. In humans, caffeine acts as a central nervous system and metabolic stimulant. Caffeine is a receptor antagonist at all adenosine receptor subtypes, which prevents feelings of drowsiness and physical fatigue. Caffeine may offer a modest protective effect against some diseases, including Parkinson's disease, cardiovascular disease and certain types of cancer. The United States Food and Drug Administration (FDA) has classified caffeine as generally recognized as safe (GRAS) as toxic doses (over 10 g/day for an average adult) are much higher than typically used doses (less than 500 mg/day). Evidence of a risk to pregnancy is equivocal, and the U.K. Food Standards Agency and the American Congress of Obstetricians and Gynecologists recommend pregnant women limit their caffeine intake to 200 mg/day. Caffeine is water soluble and is able to penetrate the skin barrier if applied topically.

Caffeine is metabolized in the liver by the cytochrome P450 oxidase enzyme system into three main metabolites: paraxanthine (1,7-dimethyl-3H-purine-2,6-dione), theobromine (3,7-dimethyl-1H-purine-2,6-dione) and theophylline (1,3-dimethyl-7H-purine-2,6-dione). Paraxanthine is the primary caffeine metabolite (84%), followed by theobromine (12%) and theophylline (4%). 1,3,7-trimethyl uric acid is a minor caffeine metabolite. The caffeine metabolites are less soluble than caffeine but retain many of its physiological effects.

### SUMMARY

In a first aspect, the invention is a kit for reducing scarring comprising a first composition comprising resveratrol and a second composition comprising a primary agent selected from the group consisting of caffeine, caffeine metabolites, and mixtures thereof.

In a second aspect, the invention is a composition for reducing scarring comprising a substance selected from the group consisting of caffeine, a caffeine metabolite, and mixtures thereof, and a pharmaceutically acceptable carrier.

In a third aspect, the invention is a method of reducing scarring comprising applying to a wound or incision a first composition comprising resveratrol; and applying to the wound or incision a second composition comprising a substance selected from the group consisting of caffeine, caffeine metabolites, and mixtures thereof.

In a fourth aspect, the invention is a method of reducing scarring comprising applying to a wound or incision a composition comprising a substance selected from the group consisting of caffeine, caffeine metabolites, and mixtures thereof. The composition is applied at least three days after the wound or incision is formed.

### Definitions

“Resveratrol” means trans-3,4′,5-trihydroxystilbene, and includes trans-3,4′,5-trihydroxystilbene salts (such as trans-resveratrol-3-sulfate), esters, amides, prodrugs and mixtures thereof.

“MCP-1 inhibitor” means a chemical or drug which reduces the activity of monocyte chemoattractant protein-1 (MCP-1). MCP-1 inhibitors include anti-MCP-1 antibodies, fragments thereof, and conjugates thereof; anti-sense oligonucleotides; ribozymes and deoxyribozymes; interfering RNA, including small interfering RNA (siRNA) such as double-stranded interfering RNA; aptamers; chemokine-binding protein (CBP); and mixtures thereof. Although resveratrol is believed to also down-regulate MCP-1, “MCP-1 inhibitor”, as used herein, does not include resveratrol.

“Innate immunity suppressor” means a chemical or drug which reduces the Toll-like receptor-3 (TLR3)-dependent expression of cytokines. Innate immunity suppressors include hyaluronic acid oligosaccharides such as hyaluronic acid tetramer; proteases from a number of viruses, such as Enterovirus 68 3C Protease, and the 3CD protease-polymerase precursor of the hepatitis A virus; aptamers to TLR3 or TRIF; anti-TLR3 antibodies; and anti-TRIF antibodies.

“Hyaluronic acid”, “hyaluronic acid oligosaccharides” and “hyaluronic acid tetramer” include the free acids, as well as the salts and esters of these compounds, such as the sodium salt.

“Caffeine” means 1,3,7-trimethylpurine-2,6-dione, and includes caffeine salts (such as caffeine citrate), esters, amides, prodrugs and mixtures thereof.

“Caffeine metabolite” means paraxanthine (1,7-dimethyl-3H-purine-2,6-dione), theobromine (3,7-dimethyl-1H-purine-2,6-dione) or theophylline (1,3-dimethyl-7H-purine-2,6-dione), and includes caffeine metabolite salts, esters, amides, prodrugs and mixtures thereof. Caffeine is not included in the term “caffeine metabolite.”

A “unit dosage” is a premeasured amount of a substance that is intended to be used in a single application.

A “multi-dosage” is a combination of multiple unit dosages. Each individual unit dosage in a multi-dosage is intended to be used in a single application.

A “primary agent” is resveratrol, caffeine, a caffeine metabolite, or mixtures thereof.

A “secondary agent” is not resveratrol, caffeine, a caffeine metabolite, or mixtures thereof.

A “prodrug” is a chemical agent that is metabolized at or proximate to the site of application and turns into the drug in question. For example, caffeine is a prodrug of paraxanthine.

A “wound” or “incision” is a cut or break in the outer layer of skin. Stretch marks or striae are not included in the terms “wound” or “incision.”

All percentages (%) are weight/weight percentages, unless stated otherwise.

### BRIEF DESCRIPTION OF THE DRAWINGS

The invention can be better understood with reference to the following drawings and description.

FIG. 1 is a first kit for reducing scarring.

FIG. 2A is a second kit for reducing scarring.

FIG. 2B is a third kit for reducing scarring.

FIG. 3A is a microscopic image of untreated wound tissue.

FIG. 3B is a microscopic image of wound tissue that has been treated with a resveratrol ester.

FIG. 4A is a photograph of a first subject's scar before excision and treatment with a composition containing caffeine.

FIG. 4B is a photograph of the first subject's scar after excision and treatment with a composition containing caffeine.

FIG. 5A is a photograph of a second subject's scar before excision and treatment with a composition containing caffeine.

FIG. 5B is a photograph of the second subject's scar after excision and treatment with a composition containing caffeine.

FIG. 6A is a photograph of a third subject's skin growth before excision and treatment with a composition containing caffeine.

FIG. 6B is a photograph of the third subject's skin growth after excision and treatment with a composition containing caffeine.

### DETAILED DESCRIPTION

Wound healing with application of resveratrol results in rapid epithelialization within 24 hours, resulting in an attenuated scar, and in some areas an almost invisible scar. However, a small attenuated scar persists and is visible with microscopic examination.

Although it is not known exactly how resveratrol reduces scarring, resveratrol up-regulates and increases the expression of a variety of agents which are involved in wound healing. One possible explanation is that resveratrol causes the over-expression of MMP-9, interleukin-8 (IL-8) and SIRT1, and increases expression of EGFR on the keratinocyte membrane and nucleus. SIRT1 may then promote differentiation, motility and proliferation of keratinocytes, and deacetylation and inactivation of p53 protein, inhibiting p53-dependent cell death from apoptosis in response to stress in human tenocytes (fibroblast-like tendon cells). SIRT1 may induce nitric oxide (NO) production, which inhibits Class I HDAC 2 from blocking growth factors including epithelial growth factor, keratinocyte growth factor 2, fibroblast growth factor 10 (FGF-10) and insulin-like growth factor 1 (IGF-1). SIRT1 may also decrease inflammation and apoptosis through a variety of mechanisms. IL-8 has a direct and profound stimulatory effect on the migration of keratinocytes, which is likely via the PLC-γ pathway. IL-8 may also recruit neutrophils. As noted above, MMP-9 degrades the Type IV collagen of the basement membrane. EGFR may cause keratinocyte and fibroblast migration and may protect and repair tissue through nuclear DNA repair. Resveratrol may also inhibit NE-KB-dependent pro-inflammatory and matrix-degrading gene products induced by IL-1β and nicotinamide.

Recent studies revealed that preventing fibroplasia during the proliferation phase of wound healing can result in little to no scar formation. Nearly scar-free healing was achieved by applying a combination of resveratrol and an MCP-1 inhibitor to wounds or incisions that did not have any injured or missing tissue that was more than 3 cm from uninjured tissue. Compositions including resveratrol and an MCP-1 inhibitor were applied either prior to formation of a wound or incision, or up to 24 hours after wound or incision formation.

Caffeine has been found to inhibit SMAD signaling and diminish CTGF expression in lung epithelial cells. Caffeine and its metabolites suppress CTGF expression in the liver via a mechanism that involves reduction of the steady state concentration of total SMAD2 protein, decreased phosphorylation of SMAD3 and up-regulation of the nuclear receptor peroxisome proliferator-activated receptor-γ (PPARγ). Paraxanthine may be a better inhibitor of CTGF expression in parenchymal cells such as liver cells.

The present invention makes use of the discovery that optionally applying a composition containing resveratrol prior to formation of a wound or incision, or up to 24 hours after wound or incision formation, followed by applying a composition containing caffeine or a caffeine metabolite during the proliferation phase of wound healing can significantly reduce or prevent the formation of scars. Resveratrol prevents fibroplasia while caffeine or a caffeine metabolite inhibits TGF-β1 production and CTGF expression.

It was surprisingly discovered that caffeine or a caffeine metabolite should be applied at least three days after a wound or incision is formed to allow the wound to begin the healing process while still preventing a scar from forming. The skin at a wound or incision site remains weak until the wound or incision has re-epithelialized. As a result, forces applied to the skin surrounding a wound or incision can be inordinately transferred to the wound or incision. These forces can increase tension in the wound or incision, which stimulates TGF-β1 and increases fibrosis, leading to scarring. Preferably, caffeine or a caffeine metabolite is applied to a wound or incision after the proliferation phase of wound healing has begun and the wound has re-epithelialized.

A composition containing resveratrol may be applied to a wound or incision at any time from prior to formation of the wound or incision up until at most one day after the formation of the wound or incision; preferably, the composition containing resveratrol is applied prior to formation of the wound or incision, up until at most 1 hour after the formation of the wound or incision; more preferably, the composition containing resveratrol is applied prior to formation of the wound or incision, up until at most 10 minutes after the formation of the wound or incision. Preferably, only a single application of the composition containing resveratrol is used. For example, the composition containing resveratrol may be applied topically to an incision site, or injected below an incision site, then the skin may be cut, optionally followed by closing the incision. For example, any deep structures which have been cut under the skin may be tied down using VICRYL® (polyglactin 910) sutures and the skin may be sutured or sealed using DERMABOND ADVANCED® topical skin adhesive or NEW-SKIN® liquid bandage. Alternatively, the composition containing resveratrol may be applied to the incision or wound after it is formed, followed by closing the wound or incision as described above. Preferably, the composition containing resveratrol is sterile.

Resveratrol may be administered to a wound or incision site by injection or as a topical composition such as a lotion, ointment, cream, gel, paste, foam, suspension, topical solution or other suitable topical form. The topical composition may contain a therapeutically effective amount of resveratrol and a pharmaceutically acceptable excipient or carrier. Resveratrol may be present at a concentration of at least 0.01 μmol/L, preferably at a concentration of at least 0.10 μmol/L, and more preferably at a concentration of at least 0.50 μmol/L. Examples of suitable resveratrol concentrations include 0.75, 0.80, 0.90, 1.0, 1.25, 1.5, 1.6, 1.7, 1.8, 1.9, 2.0, 2.1, 2.19, 2.2, 2.3, 2.4, 2.5, 2.6, 2.7, 2.8, 2.9, 3.0, 3.25, 3.5, 3.75, 4.0, 4.25, 4.5, 4.75, 5.0, 5.5, 6.0, 6.5, 7.0, 7.5, 8.0, 9.0, 10.0, 15.0, 20.0, 25.0, 30.0 and 35.0 μmol/L.

Some topical administration delivery forms, such as gels and pastes, result in a lower effective concentration of resveratrol being delivered to the wound or incision site due to interactions with the surrounding tissue. For example, the surrounding tissue may rapidly degrade the resveratrol or absorb the resveratrol. It may be necessary to increase the concentration of resveratrol to account for these interactions. Accordingly, resveratrol may be present at an increased concentration of at least 1 μmol/L, preferably at a concentration of at least 10 μmol/L, more preferably at a concentration of at least 50 μmol/L. Preferably, resveratrol is present at an increased concentration of at most 1000 μmol/L. Examples of suitable increased resveratrol concentrations include 7.5, 8.0, 9.0, 10, 12.5, 15, 16, 17, 18, 19, 20, 21, 21.9, 22, 23, 24, 25, 26, 27, 28, 29, 30, 32.5, 35, 37.5, 40, 42.5, 45, 47.5, 50, 55, 60, 65, 70, 75, 80, 90, 100, 150, 200, 250, 300, 350, 400, 450 and 500 μmol/L.

Resveratrol has a very low solubility in water, however only that portion which is dissolved in water will exert its effects. If resveratrol is applied dissolved in a hydrophobic medium it may slowly diffuse into the surrounding aqueous medium and undesirably extend the effective application time. Therefore, it is preferable that resveratrol be applied topically as a solution in an aqueous medium. For ease of application in a clinical setting, preferably the aqueous medium is a gel, paste, foam, suspension or thickened solution. Examples include aqueous compositions containing hydroxypropyl methylcellulose, high molecular weight hyaluronic acid, polyethylene glycol, agar, dextrin, pectin, trehalose, xanthan gum, polyoxyethylene alkyl ethers, chitosan, guar gum and sodium alginate. Other vehicles, adjuvants and excipients which are hydrophilic or have hydrophilic moieties, and are compatible with application into wounds, may also be used. Other pharmaceutically acceptable adjuvants, excipients and vehicles may also be included.

The composition containing resveratrol may optionally include an MCP-1 inhibitor. MCP-1 inhibitors include anti-MCP-1 antibodies, fragments thereof, and conjugates thereof; anti-sense oligonuclotides; ribozymes and deoxyribozymes; interfering RNA, including siRNA such as double-stranded interfering RNA; CBP and mixtures thereof. Preferably, the MCP-1 inhibitor is an siRNA or CBP. Although resveratrol is believed to also down-regulate MCP-1, an “MCP-1 inhibitor”, as used herein, does not include resveratrol.

A direct way to reduce the activity of MCP-1 is through protein binding. Chemokine-binding protein (CBP) is a protein secreted by parapoxviruses such as orf virus, bovine papular stomatitis virus (BPSV) and pseudocowpox virus. CBP shows high-affinity binding for human and mouse CC, CXC and C chemokines, particularly MCP-1 (CCL2), and prevents inflammatory monocyte recruitment. Binding MCP-1 blocks monocytes, which prevents the monocytes from being converted to fibrocytes and contributing to scar formation.

CBP may be produced recombinantly from parapoxvirus DNA. A general method for isolating and producing parapoxvirus proteins is described in Inder, M. K. et al., “Bovine papular stomatitis virus encodes a functionally distinct VEGF that binds both VEGFR-1 and VEGFR-2”, Journal of General Virology, vol. 88, pp. 781-791 (2007). The protein may be tagged, for example using FLAG octapeptide. Recombinant FLAG-tagged proteins may be expressed in suitable cells, such as 293-EBNA cells. The expressed protein may then be purified and/or quantified. A preferred source of CBP is BPSV strain V660. CBP activity may be assessed using an ELISA. CBP will bind to CCL2 (MCP-1), CCL3 (MIP-1a), CCL5 (RANTES), CCL19 (MIP-3β) and XCL1 (lymphotactin); will interact with CXCL2 (MIP-2) and CXCL4 (PF4); but will not bind to CXCL8 (IL-8), CXCL10 (IP10), or CXCL12 (SDF-1). The DNA sequence of the BPSV V660 gene encoding CBP has been deposited in GenBank under accession no. KM400588 and is identified as SEQ ID NO: 1 in the attached Sequence Listing. SEQ ID NO: 2 is the translated protein sequence of the CBP encoded by the gene of SEQ ID NO:1. For a general discussion of CBP isolated from BPSV V660, see Lee, S. et al., “Effect of a broad-specificity chemokine-binding protein on brain leukocyte infiltration and infarct development”, Stroke, vol. 46, pp. 537-544 (2015).

CBP may be present in the composition in a concentration of at least 0.1 micromoles/liter, preferably at least 0.5 micromoles/liter, and more preferably at least 1.0 micromoles/liter, including 0.25 to 25 micromoles/liter, 1.0 to 10 micromoles/liter, such as 1.5, 2.0, 2.5, 3.0, 3.5, 4.0, 4.5, 5.0, 5.5, 6.0, 6.5, 7.0 and 7.5 micromoles/liter. Similar concentrations of other MCP-1 inhibitors may also be used.

Alternatively, the activity of MCP-1 may be reduced by reducing its production at the site of the wound, by using a small interfering RNA (siRNA), which inhibits MCP-1 translation. When used as an inhibitor of protein expression, the siRNA includes a strand of RNA which is complementary to a portion of the mRNA transcribed from the gene for the protein. The strand of RNA must be long enough to reliably bind to the mRNA and to be specific for the mRNA of the protein. Preferably, the siRNA is 20 to 100 bases long, including 25, 27, 30 and 35 bases long. Preferably, a mixture of different siRNAs, all specific for the protein of interest, for example 2, 3 or 4 different siRNAs, is used together. Preferably, the siRNA is double-stranded, complemented with a RNA strand which non-translatable, for example a universal scrambled negative non-translatable RNA strand having the same length as the siRNA.

An especially preferred MCP-1 inhibitor is a mixture of three double strand interfering RNAs available from AMS Biotechnology (Europe) Limited (Milton Park, Abingdon UK), called “CCL2 (ID 6347) Trilencer-27 Human siRNA”. Other MCP-1 inhibitors include a mixture of three double strand interfering RNA available from Santa Cruz Biotechnology, Inc. (Dallas, Tex.), called “MCP-1 siRNA (h): sc-43913”; and an aptamer specific for MCP-1 from NOXXON Pharma AG (Berlin, Germany), called “Emapticap pegol (NOX-E36)”.

siRNA may be present in the composition in a concentration of at least 0.1 micromoles/liter, preferably at least 0.5 micromoles/liter, and more preferably at least 1.0 micromoles/liter, including 0.25 to 25 micromoles/liter, 1.0 to 10 micromoles/liter, such as 1.5, 2.0, 2.5, 3.0, 3.5, 4.0, 4.5, 5.0, 5.5, 6.0, 6.5, 7.0 and 7.5 micromoles/liter.

siRNA may be coupled to a nanoparticle or administered with an innate immunity suppressor to avoid unwanted inflammation. Suitable innate immunity suppressors include hyaluronic acid oligosaccharides such as hyaluronic acid tetramer; proteases from a number of viruses, such as enterovirus 68 3C protease, and the 3CD protease-polymerase precursor of the hepatitis A virus; aptamers to TLR3 or TRIF; anti-TLR3 antibodies; and anti-TRIF antibodies. Preferably, the innate immunity suppressor is hyaluronic acid tetramer.

Hyaluronic acid tetramer may be present in the composition containing resveratrol at a concentration of at least 1 μmol/L, preferably at a concentration of at least 10 μmol/L, more preferably at a concentration of at least 50 μmol/L. Hyaluronic acid tetramer may be present in the composition containing resveratrol at a concentration of at most 1000 μmol/L, preferably at a concentration of at most 400 μmol/L. Examples of suitable hyaluronic acid tetramer concentrations include 7.5, 8.0, 9.0, 10, 12.5, 15, 16, 17, 18, 19, 20, 21, 21.9, 22, 23, 24, 25, 26, 27, 28, 29, 30, 32.5, 35, 37.5, 40, 42.5, 45, 47.5, 50, 55, 60, 65, 70, 75, 80, 90, 100, 150, 200, 250, 300 and 350 μmol/L. Other innate immunity suppressors may be used at similar concentrations.

The composition containing resveratrol may optionally contain calcium. Calcium may be added as calcium chloride or as other calcium salts. The concentration of calcium may be at least 0.05 mmol/L, preferably at least 0.1 mmol/L. Examples of suitable calcium concentrations include 0.15 to 3.0 mmol/L or 0.2 to 1.0 mmol/L, including 0.25, 0.30, 0.35, 0.40, 0.45 and 0.5 mmol/L.

The composition containing resveratrol may optionally contain magnesium. Magnesium may be added as magnesium chloride or as other magnesium salts. The concentration of magnesium may be at least 0.5 mmol/L, preferably at least 1.0 mmol/L. Examples of suitable magnesium concentrations include 1.5 to 30 mmol/L or 2.0 to 10 mmol/L, including 2.5, 3.0, 3.1, 3.2, 3.3, 3.4, 3.5, 4.0, 4.5 and 5.0 mmol/L.

The composition containing resveratrol may optionally contain other active agents, such as other activators of SIRT1; HDAC2 (a Class I histone deacetylase) inhibitors, such as trichostatin A; agents which stimulate the production of certain growth factors such as EGF, FGF-10 and IGF-1; luteolin; tretinoin (all-trans retinoic acid); and high molecular weight hyaluronic acid.

The composition containing resveratrol may optionally contain agents that do not materially affect the basic and novel characteristics of the composition. For example, the composition containing resveratrol may optionally include agents such as stabilizers, preservatives or pH adjusters. Any additional agents must not impair the activity of the resveratrol to reduce or prevent scarring.

The composition containing resveratrol may be supplied in unit dosage form. Examples of unit dosage forms include pre-filled syringes, pouches, packets and tubes. Another example is a tube or dispenser which may be used to form foam of its contents just prior to application, for example by shaking or using a foaming agent. A self-foaming tablet, which forms foam when placed into water, may also be used. The volume of material present in the unit dosage forms may be 0.1 to 100 mL, or 1 to 50 mL, including 2, 3, 4, 5, 6, 7, 8, 9, 10, 11, 12, 13, 14, 15, 20, 25, 30, 35, 40 and 45 mL.

The composition containing resveratrol may be applied to wounds or incisions that do not have any injured or missing tissue which is more than 3 cm from uninjured tissue. Examples include almost all incisions purposefully created by a surgeon because the surgeon is able to bring the edges of the skin at the location of the incision to well within 3 cm of each other. No part of the skin surface of the wound or incision is more than 3 cm from uninjured skin, preferably no part of the skin surface of the wound or incision is more than 2 cm from uninjured skin, more preferably no part of the skin surface of the wound or incision is more than 1 cm from uninjured skin, and even more preferably no part of the skin surface of the wound or incision is more than 0.5 cm from uninjured skin.

A composition containing caffeine or a caffeine metabolite may be applied to a wound or incision during the proliferation phase of wound healing. Typically, this involves application of the composition containing caffeine or a caffeine metabolite 7 to 10 days after the wound or incision is formed. The composition containing caffeine or a caffeine metabolite may be applied as early as three days after the wound or incision is formed. Preferably, the composition containing caffeine or a caffeine metabolite is applied after any suture holes have re-epithelialized, typically about three days after the sutures are removed. Preferably, the composition containing caffeine or a caffeine metabolite is sterile.

Caffeine or a caffeine metabolite may be administered to a wound or incision site as a topical composition. The topical composition may contain a therapeutically effective amount of caffeine or a caffeine metabolite and a pharmaceutically acceptable excipient or carrier. Preferably, the pharmaceutically acceptable excipient or carrier is water-based (aqueous). The composition containing caffeine or a caffeine metabolite may be administered as a lotion, ointment, cream, gel, paste, foam, suspension, topical solution or other suitable topical form. Preferably, caffeine or a caffeine metabolite is administered as a cream. A cream may contain 0.01% to 10% caffeine or a caffeine metabolite, preferably 1% to 5% caffeine or a caffeine metabolite, including 1.0%, 1.5%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0% and 4.5% caffeine or a caffeine metabolite.

The composition containing caffeine or a caffeine metabolite may be applied to a wound or incision for a period of 1 to 12 months, including 2, 3, 4, 5, 6, 7, 8, 9, 10 or 11 months. A preferred treatment period is 3 to 6 months. The composition containing caffeine or a caffeine metabolite may be applied multiple times per day. For example, the composition containing caffeine or a caffeine metabolite may be applied once per day, twice per day, three times per day or more than three times per day. The total amount of caffeine or a caffeine metabolite applied per day may be at most 10 g/day, preferably at most 1 g/day, more preferably at most 500 mg/day. For a pregnant subject, the total amount of caffeine or a caffeine metabolite applied per day is preferably at most 200 mg/day.

The composition containing caffeine or a caffeine metabolite may be supplied in unit dosage form. Examples of unit dosage forms include tubes, packets and pouches. Another example is a tube or dispenser which may be used to form foam of its contents just prior to application, for example by shaking or using a foaming agent. A self-foaming tablet, which forms foam when placed into water, may also be used. The volume of material present in the unit dosage forms may be 0.1 to 100 mL, or 1 to 50 mL, including 2, 3, 4, 5, 6, 7, 8, 9, 10, 11, 12, 13, 14, 15, 20, 25, 30, 35, 40 and 45 mL.

Alternatively, the composition containing caffeine or a caffeine metabolite may be supplied as a multi-dosage that contains multiple unit dosages. The multi-dosage preferably contains one unit dosage for each application throughout the treatment period. For example, a 3-month (90 day) treatment period that calls for twice daily application of a composition containing caffeine or a caffeine metabolite may be supplied as a multi-dosage containing 180 unit dosages of the composition containing caffeine or a caffeine metabolite.

A composition containing caffeine or a caffeine metabolite may be supplied in a container such as a squeeze tube, jar, tub, tin, pump, spray pump, aerosol spray container, bag-on-valve container or spray bottle. The volume of the composition containing caffeine or a caffeine metabolite in the container may correspond to a specific treatment period. For example, a 4.0 oz squeeze tube may contain a one-month supply of a composition comprising caffeine or a caffeine metabolite. Dosing and application instructions may be printed on the outside of the container.

A multi-dosage may include primary agents containing caffeine, a caffeine metabolite, or mixtures thereof, and, optionally, secondary agents. The secondary agents may be pharmaceutically acceptable carriers with no biologically active agents, such as a unit dosage of carboxymethyl cellulose. The secondary agents may also be agents that promote the healing process, such as, for example, anti-inflammatory agents, antibiotics, analgesics or moisturizers.

Including secondary agents in a multi-dosage of a composition containing caffeine or a caffeine metabolite may improve patient compliance with an application protocol. Patients may unintentionally fail to comply with a recommended application protocol, for example, by forgetting to begin application or by miscounting the number of days to wait after the wound is formed before beginning application. A multi-dosage that includes secondary agents allows patients to begin applying a topical composition to a wound or incision immediately, which encourages the development of a daily application ritual. For example, a health care professional may advise a patient to apply a composition containing caffeine or a caffeine metabolite to a wound or incision once per day beginning 6 days after the wound or incision is formed as part of a 2 month (60 day) application schedule. The patient may be given a multi-dosage that contains 5 unit dosages of an antibiotic and 55 unit dosages of the composition containing caffeine or a caffeine metabolite and be instructed to begin application the first day after the wound or incision is formed.

The composition containing caffeine or a caffeine metabolite may optionally be supplied in a medicated bandage. The bandage may be, for example, an adhesive bandage, a gauze bandage, a silicone sheet or sleeve, a compression sleeve or other suitable wound dressing. The medicated bandage may contain an absorbent pad that carries the caffeine or caffeine metabolite. The medicated bandage may optionally include secondary agents that promote the healing process, such as anti-inflammatory agents, antibiotics, analgesics or moisturizers. A medicated bandage may deliver caffeine or a caffeine metabolite while also keeping the wound or incision clean and keeping the broken edges of the skin together to encourage the healing process. A single medicated bandage is equivalent to a unit dosage; multiple medicated bandages are equivalent to a multi-dosage.

The composition containing caffeine or a caffeine metabolite may optionally contain agents that do not materially affect the basic and novel characteristics of the composition. For example, the composition containing caffeine or a caffeine metabolite may optionally include agents such as stabilizers, preservatives or pH adjusters. Any additional agents must not impair the activity of the caffeine or a caffeine metabolite to reduce or prevent scarring.

A kit for reducing scarring may include a composition containing resveratrol and a composition containing caffeine or a caffeine metabolite. The composition containing resveratrol may be supplied as a unit dosage while the composition containing caffeine or a caffeine metabolite may be supplied as a multi-dosage. The kit may optionally include unit dosages of secondary agents in the multi-dosage. Preferably, the composition containing resveratrol is administered by a healthcare professional, such as a doctor, nurse or medical technician. Preferably, the composition containing caffeine or a caffeine metabolite and any unit dosages of secondary agents are self-administered by the subject.

The kit may optionally include printed instructions. There may be two sets of printed instructions in the kit, a first set of printed instructions explaining the use of the composition containing resveratrol and a second set of printed instructions explaining the use of the composition containing caffeine or a caffeine metabolite.

The kit may optionally include a topical antiseptic. For example, the antiseptic may be delivered in a swab, wipe, gel, ointment or spray. The antiseptic may be used to clean the wound or incision site prior to administering the composition containing resveratrol.

The kit may optionally include a container for housing the kit ingredients. The container protects the compositions and other ingredients from damage during transport. Preferably, the container is formed of a rigid, durable material such as plastic.

The kit may be divided into two portions, a physician portion and a patient portion. The physician portion may include a unit dosage of a composition containing resveratrol and, optionally, printed instructions, sutures, topical skin adhesives, liquid bandages and/or antiseptics. The patient portion may include a multi-dosage of a composition containing caffeine or a caffeine metabolite and, optionally, printed instructions. The multi-dosage may optionally include unit dosages of secondary agents. The patient portion may be packaged in its own container within the kit so that it may be sent home with a patient for self-application.

A kit for reducing scarring may be offered in different formulations for different intended subjects. For example, a kit for reducing scarring that is to be used by an adult male subject may be formulated to deliver 500 mg of caffeine or a caffeine metabolite per day. Alternatively, a kit for reducing scarring that is to be used by a pregnant adult female subject may be formulated to deliver 200 mg of caffeine or a caffeine metabolite per day.

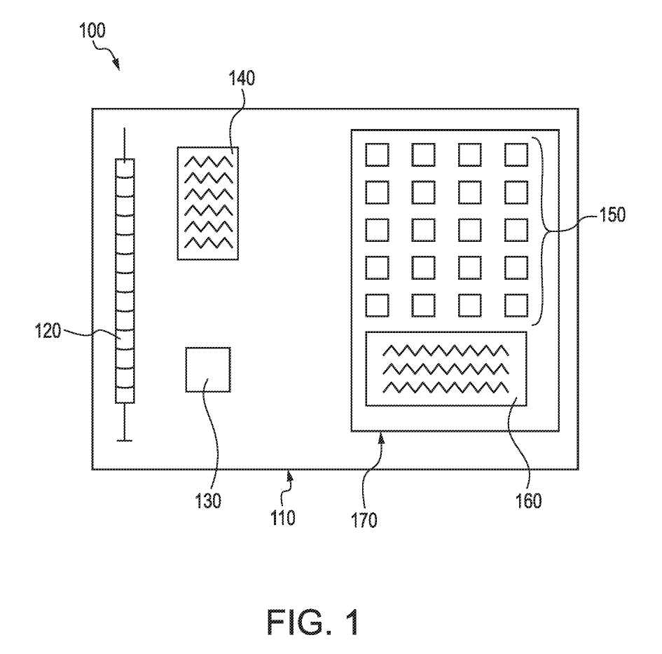

FIG. 1 shows a kit for reducing scarring 100. The kit is supplied in a rigid container 110. The kit includes a unit dose of a composition comprising resveratrol 120 packaged in a syringe. An antiseptic 130 is supplied in the form of an alcohol swab. A first set of instructions 140 explains how to administer the composition comprising resveratrol. The kit includes a multi-dosage of a composition comprising caffeine or a caffeine metabolite 150 packaged in multiple single-use pouches. A second set of instructions 160 explains how to administer the composition comprising caffeine or a caffeine metabolite. The multi-dosage of the composition comprising caffeine or a caffeine metabolite and the second set of instructions are contained in a package 170 within the kit.

Alternatively, a kit for reducing scarring may include a multi-dosage of a composition containing caffeine or a caffeine metabolite and, optionally, printed instructions. The multi-dosage may optionally include unit dosages of secondary agents. The composition containing caffeine or a caffeine metabolite may be packaged in multiple unit dosages or in a single suitable container.

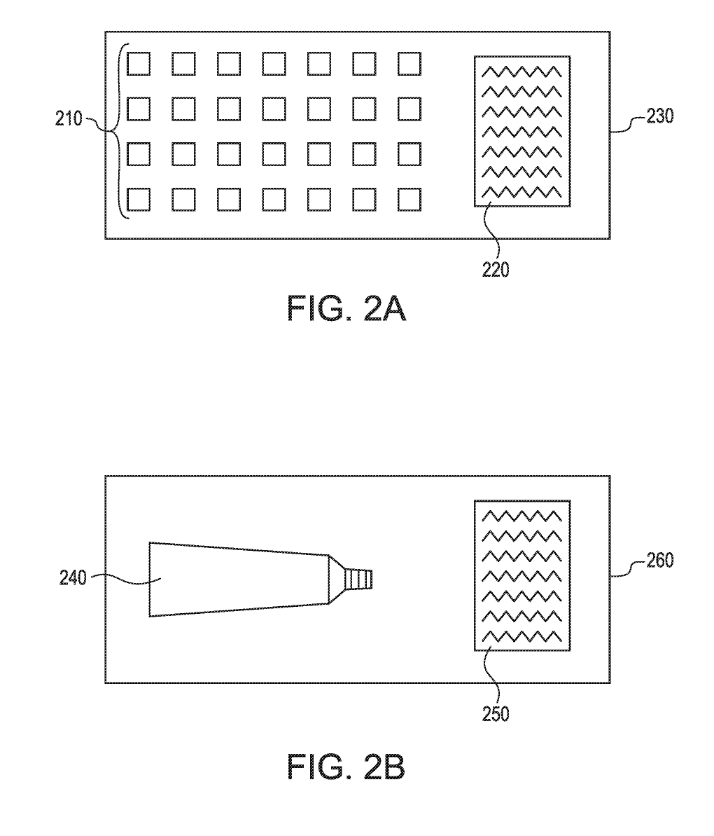

FIG. 2A and FIG. 2B show alternative designs of kits for reducing scarring that include a one-month supply of a composition containing caffeine or a caffeine metabolite. FIG. 2A shows a kit for reducing scarring that includes 28 individual unit dosages of a composition containing caffeine or a caffeine metabolite 210 and printed instructions 220 in a rigid container 230. FIG. 2B shows a kit for reducing scarring that includes a tube containing a one-month supply of a composition containing caffeine or a caffeine metabolite 240 and printed instructions 250 in a rigid container 260.

### EXAMPLES

### Example 1—Resveratrol and Caffeine Treatment

Human subjects undergoing a surgical procedure involving an incision were included in a study to reduce or prevent scar formation. A composition containing resveratrol and siRNA was instilled at the time of incision on the incision site. 2 weeks post-incision caffeine was applied to the incision site using silicone sheeting. The caffeine application was continued for 3 months. The resveratrol/caffeine treatment was found to block scar formation that starts 2-3 weeks after incision or wound formation.

### Example 2—Resveratrol Ester Histology

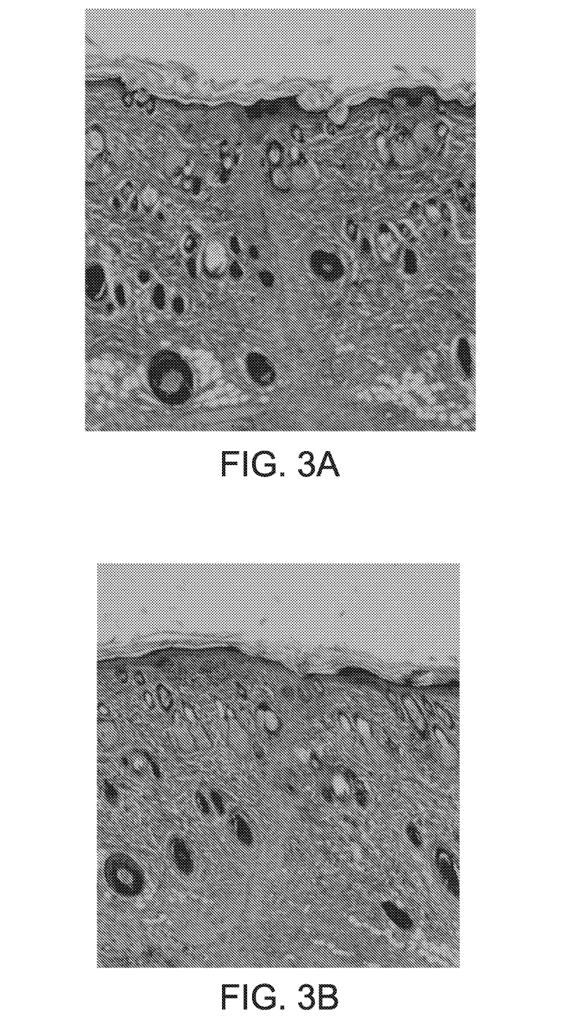

Histological studies of treatment with a resveratrol ester revealed a visible decrease in monocytes and fibroblasts in the healing junction. FIG. 3A is a microscopic image of an untreated control incision. FIG. 3B is a microscopic image of an incision treated with a resveratrol ester. The treated incision showed notably uniform epidermal repair as compared to the deep indention of the epidermis seen in the untreated control incision. It was estimated that treatment with the resveratrol ester resulted in a 40-50% decrease in cellular scar/fibrosis components.

### Example 3—Caffeine Cream for Scar Reduction or Prevention

The ability of caffeine to reduce or prevent scar formation was studied. Two subjects with existing scars were chosen for the study. The existing scars were surgically removed and the subjects were instructed to apply a cream containing caffeine to the incision.

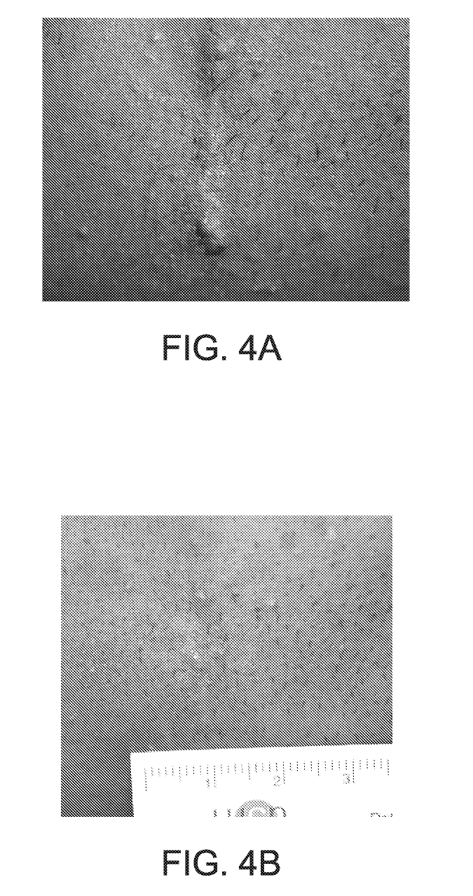

Subject #1 presented with a skin growth on the left mid cheek. FIG. 4A shows the initial skin growth. Caffeine cream application began approximately 3 weeks after the excision of the skin growth. A significant subdermal scar was palpable at the time the subject began applying the caffeine cream. A good decrease in scarring was noted and a softer, nearly resolved deep scar was palpable after 8 weeks of application of the caffeine cream. FIG. 4B shows the incision site after 8 weeks of caffeine cream application.

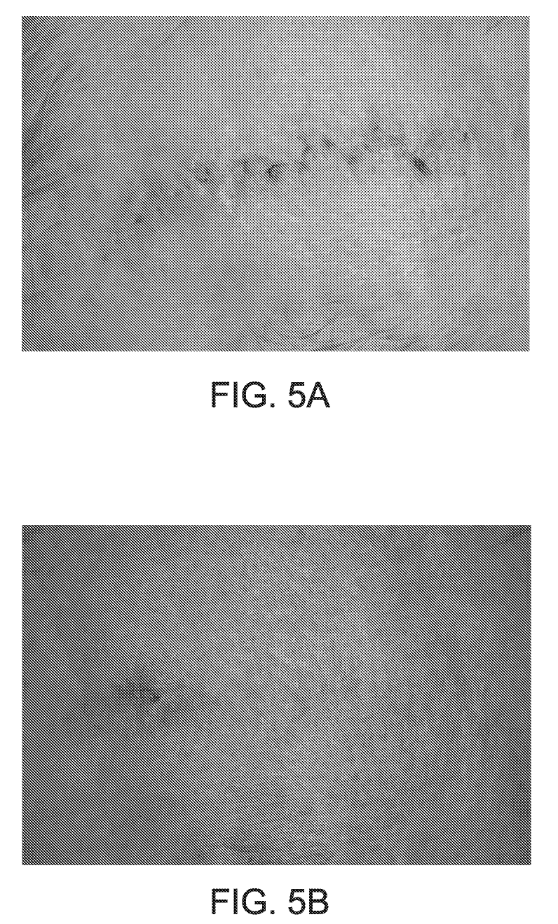

Subject #2 presented with an older mid-forehead scar. The scar had been revised twice previously with poor results. The subject underwent a third revision of the scar. FIG. 5A shows the incision site one week after incision immediately following suture removal. The subject began applying the caffeine cream immediately after suture removal and continued for two months. A very small, if not invisible, scar was noted after 7 weeks of caffeine cream application, as shown in FIG. 5B. It was also noted that the subject had small punctate areas where the suture holes were located. It appeared that application of the caffeine cream prevented the healing of two suture puncture sites that were formed during excision, which had become small punctate erosive areas.

The study showed that postsurgical caffeine cream application starting 1 week after incision or wound formation can inhibit scarring. The caffeine was rapidly taken from the application site and distributed throughout the body, as both subjects felt caffeine effects. Nighttime application of the caffeine cream was found to cause caffeine insomnia, indicating that the caffeine is absorbed through the skin and travels beyond the treatment site. It was theorized that treatment site incisions act as pools for topically applied molecules and transport the molecules to the bloodstream quicker than normal skin.

### Example 4—Caffeine Cream for Scar Reduction or Prevention

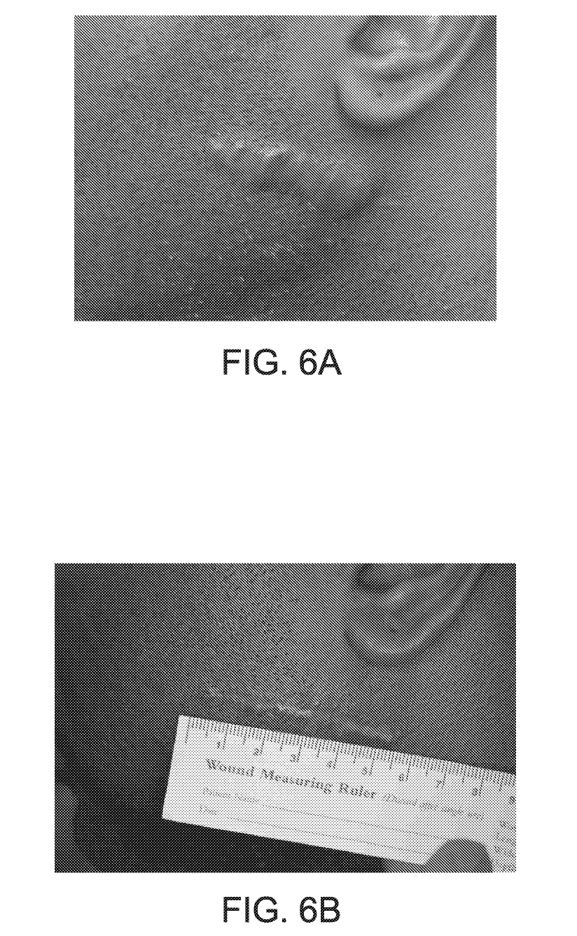

An adult male subject present with a skin growth on his left cheek that had previously been excised twice before. The most recent excision was performed approximately 12 months ago and was followed with postoperative radiation therapy. FIG. 6A shows the skin growth after two previous excisions. The skin growth was deep and rapidly rising. The subject underwent a third excision of the skin growth. A 3% caffeine cream was applied starting 7 days after excision and one day after sutures were removed from the incision site. The subject was instructed to apply the 3% caffeine cream twice daily for 6 months. FIG. 6B shows the skin growth site 3 weeks after excision and treatment with caffeine cream. The skin growth site was much softer with only a minimal deep firm scar component remaining.

### Example 5—Resveratrol and Caffeine/Caffeine Metabolite Treatment (Prophetic)

Human subjects undergoing a surgical procedure involving an incision will have a composition containing an effective amount of resveratrol and an effective amount of siRNA instilled at the time of incision on the incision site. 3 days post-incision a composition containing 3% caffeine in CETAPHIL® will be applied to the incision site. Application of the composition containing caffeine will continue for 3 months. A caffeine metabolite, such as paraxanthine, may be used instead of caffeine in the CETAPHIL®-based composition.

### Example 6—Caffeine/Caffeine Metabolite Treatment (Prophetic)

A cream will be formulated as 3% caffeine with CETAPHIL® as a water-soluble base. The cream will be applied to an incision or wound after the wound or incision is re-epithelialized. Typically, this will be about 7-10 days after the incision or wound is formed. It may be desirable to wait to begin application until after any suture holes have re-epithelialized. The caffeine cream will be applied twice daily for 3 to 6 months. A caffeine metabolite, such as paraxanthine, may be used instead of caffeine in the CETAPHIL®-based composition.

What is claimed is:

1. A kit for reducing scarring, comprising:
a first composition comprising resveratrol, and
a second composition comprising a primary agent selected from the group consisting of caffeine, caffeine metabolites, and mixtures thereof.

2. The kit of claim 1, wherein the first composition further comprises an MCP-1 inhibitor.

3. (canceled)

4. The kit of claim 1, wherein the first composition further comprises an innate immunity suppressor.

5-7. (canceled)

8. The kit of claim 1, wherein the second composition comprises a caffeine metabolite.

9. The kit of claim 8, wherein the caffeine metabolite is paraxanthine.

10-14. (canceled)

15. The kit of claim 1, wherein the first composition further comprises an MCP-1 inhibitor, an innate immunity suppressor, calcium and magnesium, and
the second composition comprises a caffeine metabolite.

16. The kit of claim 1, further comprising a third composition, wherein the third composition comprises a secondary agent selected from the group consisting of pharmaceutically acceptable carriers, anti-inflammatory agents, antibiotics, analgesics and moisturizers.

17. (canceled)

18. A composition for reducing scarring, comprising:
a substance selected from the group consisting of caffeine, a caffeine metabolite, and mixtures thereof, and
a pharmaceutically acceptable carrier.

19. The composition of claim 18, wherein the composition comprises the caffeine metabolite.

20. (canceled)

21. (canceled)

22. A method of reducing scarring, comprising:
applying to a wound or incision a first composition comprising resveratrol; and
applying to the wound or incision a second composition comprising a substance selected from the group consisting of caffeine, caffeine metabolites, and mixtures thereof.

23. (canceled)

24. The method of claim 22, wherein the second composition is applied at least seven days after the wound or incision is formed.

25. (canceled)

26. The method of claim 22, wherein the second composition is applied after any suture holes have re-epithelialized.

27. The method of claim 22, wherein the first composition is applied within one hour of the wound or incision being formed.

28-29. (canceled)

30. The method of claim 22, wherein no part of the surface of the wound or incision is more than 3 cm from uninjured skin.

31-36. (canceled)

37. The method of claim 22, wherein the first composition further comprises an MCP-1 inhibitor,
the second composition comprises paraxanthine,
the first composition is applied before the wound or incision is formed, or up to 24 hours after the wound or incision is formed,
the second composition is applied at least three days after the wound or incision is formed, and
no part of the surface of the wound or incision is more than 3 cm from uninjured skin.

38. A method of reducing scarring, comprising:
applying to a wound or incision the composition of claim 18,
wherein the composition is applied at least three days after the wound or incision is formed.

39. (canceled)

40. The method of claim 38, wherein the composition comprises the caffeine metabolite.

41. The method of claim 40, wherein the caffeine metabolite is paraxanthine.

42. The kit of claim 15, wherein the MCP-1 inhibitor comprises siRNA, the innate immunity suppressor comprises a hyaluronic acid tetramer, and the caffeine metabolite comprises paraxanthine.


SEQUENCE LISTING (2 sequences)

SEQ ID NO: 1 - DNA, 897 bp, Bovine papular stomatitis virus strain V660
atgaagtcgacatatctgttattcgcgttatccttagtattatcagcggcactgagcgcgccgatgaacacagacgaacaaaggaactggtgtagaattcatcacgacaagctctattctagatttagactttatatgaagataactagaacaacagttacgccaccgacgggaatgtgcatgctggacatagaaaactcaaacatggatctagatgagactgatgaagttaccggcgacactagcaaaatttccttcagggattctacagcaacgttcgaagcatctggcataaatggaagcataacactgatgggggacgaagttcatatacccatgagttacatcggtgtcagttttgacccttccagtgactatgtgttcgttaacgtgtcttcctggactccgtgggaaaaatctaccactgatttggcagccaatggctttgagggtctaaaaaatattatcaaccaccaaatattggctattaaccttggatgcgaccatcagtacccggcgcccaatcctacaccaaagccggtaaagattagtacaccaagcaccacacctggtccgactcttctggaaactccagaggcacctactaccgtttctcctgtggacacgtcgtccgaccgaaagaagaatcctgatcctgtcgacttctcgttgctagtagataccagatgcataaagaacgtggatgtgtactttgaaattaaggacgcgtgtctggcgtataagagccagagtccatttaagttggtaggacaacatggaacaggccagcaagtgcgtcaggagatgaagagtttgggaacctcctataggacatgcaagttaaaaaccagttccactgcagacaacgacatggaatccgcaaaggatgaggagtaa

SEQ ID NO: 2 - protein, 298 aa, Bovine papular stomatitis virus strain V660
MKSTYLLFALSLVLSAALSAPMNTDEQRNWCRIHHDKLYSRFRLYMKITRTTVTPPTGMCMLDIENSNMDLDETDEVTGDTSKISFRDSTATFEASGINGSITLMGDEVHIPMSYIGVSFDPSSDYVFVNVSSWTPWEKSTTDLAANGFEGLKNIINHQILAINLGCDHQYPAPNPTPKPVKISTPSTTPGPTLLETPEAPTTVSPVDTSSDRKKNPDPVDFSLLVDTRCIKNVDVYFEIKDACLAYKSQSPFKLVGQHGTGQQVRQEMKSLGTSYRTCKLKTSSTADNDMESAKDEE